(19) United States
(12) Patent Application Publication
(10) Pub. No.: US 2023/0417750 A1
(43) Pub. Date: Dec. 28, 2023

(54) SINGLE-MOLECULE ELECTRONIC MULTIPLEX NANOPORE IMMUNOASSAYS FOR BIOMARKER DETECTION

(71) Applicant: The Trustees of Columbia University in the City of New York, New York, NY (US)

(72) Inventors: Jingyue Ju, Englewood Cliffs, NJ (US); Shiv Kumar, Belle Mead, NJ (US); James J. Russo, New York, NY (US); Chuanjuan Tao, Fort Lee, NJ (US); Steffen Jockusch, New York, NY (US); Xiaoxu Li, New York, NY (US)

(73) Assignee: The Trustees of Columbia University in the City of New York, New York, NY (US)

(21) Appl. No.: 18/339,717

(22) Filed: Jun. 22, 2023

Related U.S. Application Data
(63) Continuation of application No. PCT/US2021/065050, filed on Dec. 23, 2021.
(60) Provisional application No. 63/130,402, filed on Dec. 23, 2020.

(51) Int. Cl. G01N 33/569; G01N 33/537

(52) U.S. Cl. CPC G01N 33/56983; G01N 33/537; G01N 2333/165; G01N 2469/20; G01N 2469/10

(57) ABSTRACT

This invention provides methods for detecting viruses, viral antigens, viral antibodies, and other antigens and antibodies using single molecule electronic nanopores and polymer tags.

This application claims the benefit of U.S. Provisional Application No. 63/130,402, filed Dec. 23, 2020, the entirety of which is hereby incorporated by reference.

Throughout this application, certain patents and publications are referenced, the latter by authors and publication year. Full citations for these publications may be found immediately preceding the claims. The disclosures of these patents and publications in their entireties are hereby incorporated by reference into this application in order to describe more fully the state of the art to which this invention relates.

### FIELD OF INVENTION

This invention provides methods for detecting viruses, viral antigens, viral antibodies, and other antigens and antibodies using single molecule electronic nanopores and polymer tags.

### BACKGROUND OF THE INVENTION

Classic immunological approaches to detection of proteins using antibodies, receptors, or other binding partners include, among others, enzyme-linked immunosorbent (ELISA) assay (generally in the form of an antibody sandwich method), radioimmunoassays, and immunoblotting methods²⁴⁻²⁶, with equivalent biochemical approaches being used for protein-receptor and protein-ligand reactions. Majority of these methods rely on examining one protein at a time. Moreover, the protein target typically must be present in large amounts and at relatively high concentrations to assure a reliably detectable signal.

Beginning in 1975 but largely over the last 15 years, methods that attempt to simultaneously examine many different proteins have appeared. These include 2-D gel electrophoresis, tandem mass spectrometry (MS-2 or MS-3) systems with intermediate protein cleavage, isotope-coded affinity tag (ICAT)-MS, MudPIT (LC-2/MS-2), and combinations of these approaches²¹⁻³⁰. For instance, in the ICAT approach, proteins from different tissues are labeled with tags containing either hydrogen or deuterium, and the differential patterns are observed by mass spectrometry. While many of these methods allow multiple samples to be compared concurrently, due to the cost of associated technologies such as mass spectroscopy, many of these approaches have not found general utility.

A relatively recent addition to the repertoire, protein arrays, in which fluorescently labeled proteins are allowed to bind to numerous spots, each containing covalently attached antibodies for a specific protein (antigen), are an appealing solution, as they can be mass produced and data analysis standardized⁴⁰⁻⁴³. While limited by the number of available specific antibodies that can function on a solid phase, a more important shortcoming of this method is the relative binding ability of the antibodies. Unlike DNA probes on gene expression microarrays, where probes can be selected to be fairly uniform in their binding affinity for mRNA targets, different antibodies may bind their fluorescently labeled antigens with very different affinities. Because of this variable affinity, quantification from spot to spot (antigen to antigen) becomes difficult, especially when combined with the likelihood that the fluorescent signal can lie outside of the linear range of detection. For example, low copy number proteins in the sample will not be seen, unless their binding is stronger than the average antigen-antibody interaction elsewhere on the chip, in which case they will be over-represented. Moreover, the effective concentration range will have the same floors and ceilings as other fluorescent methods on microarrays, and small changes in protein levels will be difficult to distinguish using protein arrays. Another common issue with fluorescent labeling is the existence of overlapping emission spectra, which limits the number of differentially labeled samples that can be applied to the arrays.

In summary, gene regulation analysis at the level of protein synthesis, like proteomics in general, lags behind nucleic acid analysis in its throughput, sensitivity and automation. This is due to the relatively poor stability of proteins, their high heterogeneity, and the requirement for a much wider dynamic range of detection with increased demand for sensitivity approaching the single molecule detection level, a need not easily met by fluorescent or colorimetric measurements. While protein arrays based on antibody interactions with fluorescently labeled antigens or secondary antibodies have gained some degree of popularity over the last decade⁴², some of the drawbacks of this approach, including fluorescence saturation and overlap in fluorescent emission, make accurate quantification difficult. While there are currently several examples of application of nanopore-based analytics, no existing technology allows quantification of protein-protein interactions with a plurality of tags.

### DETAILED DESCRIPTION OF THE INVENTION

This invention provides methods for detecting viruses, viral antigens, viral antibodies, and other antigens and antibodies using single molecule electronic nanopores and polymer tags.

In an embodiment of the invention, the tag moiety comprises more than one detectable component. It is contemplated that each such detectable component is independently detectable.

In a further embodiment of the invention, the detectable component is selected from the group consisting of ethylene glycol, an amino acid, a carbohydrate, a peptide, a dye, a fluorescent compound, a chemilluminiscent compound, a mononucleotide, a dinucleotide, a trinucleotide, a tetranucleotide, a pentanucleotide, a hexanucleotide, a polynucleotide, a nucleotide monophopshate, a nucleotide diphosphate, a nucleotide polyphosphate, an aliphatic acid, an aromatic acid, an unsubstituted alcohol or thiol, an alcohol or a thiol substituted with one or more halogens, a cyano group, a nitro group, an alkyl group, an alkenyl group, an alkynyl group, and an azido group.

In certain embodiments, the detectable component of said tag moieties comprises a multiplicity of ethylene glycol units. In a further embodiment, the multiplicity of ethylene glycol units comprises 16, 20, 24, or 36 ethylene glycol units.

In an embodiment of the invention disclosed herein, the tag moiety attaches to the compound of interest via a cleavable linker. In further embodiments of the invention, the cleavable linker is a photocleavable linker or a chemically cleavable linker.

In one embodiment, the photocleavable linker is a 2-nitrobenzyl linker. In another embodiment, the chemically cleavable linker is an azido linker. In an embodiment, UV light is used to cleave the photocleavable linker. Methods for production of cleavably capped and/or cleavably linked molecules are disclosed in U.S. Pat. No. 6,664,079, which is hereby incorporated by reference.

In an embodiment of the claimed method, at least one compound of interest is a protein and at least one of said tag moieties attaches to the carboxy or amino terminus of said protein.

In another embodiment, at least one compound of interest is a protein and at least one of said tag moieties attaches to a lysine, an arginine, or a cysteine residue of said protein.

In a further embodiment of invention, the nanopore is a biological nanopore, a modified biological nanopore, or a synthetic nanopore. In certain embodiments, the nanopore is proteinaceous, in particular an alpha hemolysin (α-hemolysin).

In yet another embodiment, the nanopore is a solid-state nanopore. In a specific embodiment, the nanopore comprises graphene. It is contemplate that in certain embodiment the nanopore is in a membrane.

In an embodiment of the invention, the nanopore is part of an array of nanopores. In certain embodiments, each nanopore in said array comprises identical means for binding the detectably tagged compounds of interest. In certain other embodiments, each nanopore in said array comprises different means for binding the detectably tagged compounds of interest.

In an embodiment, the means for binding the detectably tagged compounds of interest is a protein, in particular an antibody. In another embodiment, the means for binding the detectably tagged compounds of interest is non-proteinceous.

In an embodiment of the methods disclosed herein, a tag moiety is distinguishable from any other tag moiety based on blockade signature of said tag moiety detectable with said at least one nanopore. In certain embodiments, the blockade signature is result of a change in current amplitude or conductance of said at least one nanopore.

In an embodiment, said at least one nanopore further comprising a mean for ejecting said tag moiety from the nanopore.

Each method and process described herein can be performed using compound with cleavable or noncleavable tags.

For the foregoing embodiments, each embodiment disclosed herein is contemplated as being applicable to each of the other disclosed embodiments.

As used herein, and unless stated otherwise, each of the following terms shall have the definition set forth below.

“Antibody” shall include, without limitation, (a) an immunoglobulin molecule comprising two heavy chains and two light chains and which recognizes an antigen; (b) a polyclonal or monoclonal immunoglobulin molecule; and (c) a monovalent or divalent fragment thereof. Immunoglobulin molecules may derive from any of the commonly known classes, including but not limited to IgA, secretory IgA, IgG, IgE and IgM. IgG subclasses are well known to those in the art and include, but are not limited to, human IgG1, IgG2, IgG3 and IgG4. Antibodies can be both naturally occurring and non-naturally occurring. Furthermore, antibodies include chimeric antibodies, wholly synthetic antibodies, single chain antibodies, and fragments thereof. Antibodies may be human or nonhuman. Antibody fragments include, without limitation, Fab fragments, Fv fragments and other antigen-binding fragments.

“Nanopore” includes, for example, a structure comprising (a) a first and a second compartment separated by a physical barrier, which barrier has at least one pore with a diameter, for example, of from about 1 to 10 nm, and (b) a means for applying an electric field across the barrier so that a charged molecule such as DNA, nucleotide, nucleotide analogue, or tag, can pass from the first compartment through the pore to the second compartment. The nanopore ideally further comprises a means for measuring the electronic signature of a molecule passing through its barrier. The nanopore barrier may be synthetic or naturally occurring in part. Barriers can include, for example, lipid bilayers having therein α-hemolysin, oligomeric protein channels such as porins, and synthetic peptides and the like. Barriers can also include inorganic plates having one or more holes of a suitable size. Herein “nanopore”, “nanopore barrier” and the “pore” in the nanopore barrier are sometimes used equivalently. It is understood that the electric field of a nanopore may be adjustable. It is also understood that a charged molecule such as DNA, nucleotide, nucleotide analogue, or tag, does not need to pass from the first compartment through the pore to the second compartment in order to produce an electronic signature. Such electronic signature may be produced by localization of the molecule within the pore.

Nanopore devices are known in the art and nanopores and methods employing them are disclosed in U.S. Pat. Nos. 7,005,264 B2; 7,846,738; 6,617,113; 6,746,594; 6,673,615; 6,627,067; 6,464,842; 6,362,002; 6,267,872; 6,015,714; 5,795,782; and U.S. Publication Nos. 2004/0121525, 2003/0104428, and 2003/0104428, each of which are hereby incorporated by reference in their entirety.

“Blockade signature” of a molecule passing through a pore via application of an electronic field shall include, for example, the duration of the nucleotide's passage through the pore together with the observed amplitude of current during that passage. Blockade signature for a molecule is envisioned and can be, for example, a plot of current (e.g. pA) versus time for the molecule to pass through the pore via application of an electric field. Alternatively, blockade signature is also determinable for a molecule which does not pass through a pore. Blockade signature of such a molecule is also envisioned and can be, for example, a plot of current (e.g. pA) versus time for the molecule to enter into or pass adjacent to the pore. Herein “blockade signature”, “blockade signal”, and “electronic signature” are sometime used equivalently.

A specific event diagram is constructed which is the plot of translocation time versus blockade current. This specific event diagram (also referred to as an blockade signature) is used to distinguish molecules by single-channel recording techniques based on characteristic parameters such as translocation current, translocation duration, and their corresponding dispersions in the diagram.

As used herein, a “tag” or a “tag moiety” is any chemical group or molecule that is capable of producing a unique blockade signature detectable with a nanopore. In some cases, a tag comprises one or more of ethylene glycol, an amino acid, a carbohydrate, a peptide, a dye, a fluorescent compound, a chemilluminiscent compound, a mononucleotide, a dinucleotide, a trinucleotide, a tetranucleotide, a pentanucleotide, a hexanucleotide, a polynucleotide, a nucleotide monophopshate, a nucleotide diphosphate, a nucleotide polyphosphate, an aliphatic acid, an aromatic acid, an unsubstituted alcohol or thiol, an alcohol or a thiol substituted with one or more halogens, a cyano group, a nitro group, an alkyl group, an alkenyl group, an alkynyl group, an azido group, or a combination thereof.

As used herein, unless otherwise specified, a tag moiety which is different or distinguishable from the tag moiety of a referenced molecule means that the tag moiety has a different chemical structure from the chemical structure of the other/referenced tag moiety. A tag moiety is different or distinguishable from the tag moiety of a referenced molecule could also mean that the tag moiety has a different blockade signature from the blockade signature of the other/referenced tag moiety.

As used herein, a tag which “localizes” within a pore is a tag located inside or adjacent to the pore. A tag which localizes within a pore does not necessarily pass through or translocate the pore.

As used herein, “proteinaceous” compound means any biopolymer formed from amino acids, such as peptides, proteins, antibodies, antigens, or a fragment or portion thereof. Such compound may be naturally occurring or non-naturally occurring.

As used herein, “alkyl” includes both branched and straight-chain saturated aliphatic hydrocarbon groups having the specified number of carbon atoms and may be unsubstituted or substituted. Thus, C1-Cn as in “C1-Cn alkyl” includes groups having 1, 2, . . . , n−1 or n carbons in a linear or branched arrangement. For example, a “C1-C5 alkyl” includes groups having 1, 2, 3, 4, or 5 carbons in a linear or branched arrangement, and specifically includes methyl, ethyl, n-propyl, isopropyl, n-butyl, t-butyl, and pentyl

As used herein, “alkenyl” refers to a non-aromatic hydrocarbon group, straight or branched, containing at least 1 carbon to carbon double bond, and up to the maximum possible number of non-aromatic carbon-carbon double bonds may be present, and may be unsubstituted or substituted. For example, “C2-C5 alkenyl” means an alkenyl group having 2, 3, 4, or 5, carbon atoms, and up to 1, 2, 3, or 4, carbon-carbon double bonds respectively. Alkenyl groups include ethenyl, propenyl, and butenyl.

The term “alkynyl” refers to a hydrocarbon group straight or branched, containing at least 1 carbon to carbon triple bond, and up to the maximum possible number of non-aromatic carbon-carbon triple bonds may be present, and may be unsubstituted or substituted. Thus, “C2-C5 alkynyl” means an alkynyl group having 2 or 3 carbon atoms and 1 carbon-carbon triple bond, or having 4 or 5 carbon atoms and up to 2 carbon-carbon triple bonds. Alkynyl groups include ethynyl, propynyl and butynyl.

The term “substituted” refers to a functional group as described above such as an alkyl, or a hydrocarbyl, in which at least one bond to a hydrogen atom contained therein is replaced by a bond to non-hydrogen or non-carbon atom, provided that normal valencies are maintained and that the substitution(s) result(s) in a stable compound. Substituted groups also include groups in which one or more bonds to a carbon(s) or hydrogen(s) atom are replaced by one or more bonds, including double or triple bonds, to a heteroatom. Non-limiting examples of substituents include the functional groups described above, and for example, N, e.g. so as to form —CN.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

In the compound structures depicted herein, hydrogen atoms, except on ribose and deoxyribose sugars, are generally not shown. However, it is understood that sufficient hydrogen atoms exist on the represented carbon atoms to satisfy the octet rule.

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention.

All combinations of the various elements described herein are within the scope of the invention. All sub-combinations of the various elements described herein are also within the scope of the invention.

This invention will be better understood by reference to the Experimental Details which follow, but those skilled in the art will readily appreciate that the specific experiments detailed are only illustrative of the invention as described more fully in the claims which follow thereafter.

Single Molecule Electronic Detection of Viruses, Viral Antigens and Antibodies

To date, no approach has been developed to rapidly and directly detect SARS-CoV-2 or any other viral particles present at minimal environmental levels without amplification. PCR-based methods require sample preparation and take >15 min, even with substantial virus abundance; antibody-based methods are rapid, but are limited to high virus abundance. We describe a novel method that will allow rapid and direct detection of low abundance virus in environmental (air, waste water, surface) or human (sputum, nasal) samples. Our approach integrates 3 unique features: (1) the single molecule (SM) electronic detection sensitivity of nanopores, (2) the size differences between the capture agent and the viral target which allows their discrimination by nanopore (e.g., antibody vs virus particle which can be discriminated in ˜20 nm nanopores; nanobody vs viral antigen or viral particle which are discriminable in ˜5 nm nanopores; synthetic viral epitope vs viral antibody which are discriminable in ˜3 nm nanopores), and (3) DNA tagged capture agent-target molecular affinity enabling specific viral target identification. For this virus detection platform, we use a SM electronic platform with solid-state, protein or hybrid nanopores, and polymer tags, similar to that we have successfully developed for DNA/RNA sequencing,¹⁻³ genotyping^{4,5} and biomarker detection.⁶ In those cases, multiple nanopore-differentiable tagged nucleotides, tagged oligonucleotides or tagged antibodies were used to permit specificity and multiplexing. Thanks to the flexibility afforded by the use of a wide variety of polymer tags that can differentially block ionic currents in the nanopore, the invention described herein can also be expanded to detect multiple targets simultaneously, including viral antigens from different viruses and different viral antibodies from the same virus as exemplary cases, though antibodies and antigens for bacterial infections, cancer or other disease biomarkers, among other examples, can be identified and distinguished using the same approach described herein.

This platform permits very rapid electronic virus detection at the single-molecule level, matching or exceeding the current methods with regard to sensitivity and accuracy, while adding flexibility and ease of operation and analysis, and reducing cost. This approach can incorporate antibodies for a host of infectious disease viruses, including SARS-CoV-2 and influenza viruses, or future emerging viruses, as well as nanobodies for the equivalent viral antigens and synthetic antigenic epitopes for the antibodies induced during infections by these viruses. The invention described herein can be used to directly monitor air (aerosols) or surface (fomite) samples in factories, schools, hospitals, nursing homes, public transportation networks, stores, eating and drinking establishments, cruise ships, military bases and other high transmission locales, and to carry out rapid tests on sputum, nasal or blood samples in clinical or outpatient settings. By simply changing the DNA-tagged capture agent to one that can recognize surface proteins or antibodies specific to additional pathogens, monitoring of any virus or other infectious organisms can be achieved. The approach can also be used to identify emerging pathogens and weaponized viruses used in biowarfare, as well as cancer or other disease antigens or antibodies.

### BRIEF DESCRIPTION OF THE DRAWINGS

FIG. 1: Left: CMOS nanopore array chip used for SM DNA sequencing. Right: SM sequencing data using the nanopore array chip and 4 polymer tags.²

FIG. 2: The approach for SM electronic detection of coronavirus.

FIG. 3: Test assay using SARS-CoV-2 mimic consisting of Streptavidin with four conjugated Spike proteins.

FIG. 4: Synthesis of DNA tagged antibody using SiteClick method. The resulting tagged antibody is used to capture the virus or viral antigen, and can also be used in some cases to distinguish which virus or antigen is captured by using different nanopore-detectable tags.

FIG. 5: Synthesis of DNA tagged antibody using the homobifunctional linker disuccinimidyl suberate (DSS).

FIG. 6: SM electronic detection of SARS-CoV-2 Spike protein.

FIG. 7: SM electronic detection of SARS-CoV-2 Spike protein antibodies.

FIG. 8: Conjugation of synthetic epitope to polymer tag via Click chemistry reaction.

FIG. 9: Examples of Click chemistry-based conjugation reactions.

FIG. 10: Modified deoxythymidine phosphoramidites available from Glen Research.

FIG. 10 (continued): Modified deoxythymidine phosphoramidites available from Glen Research.

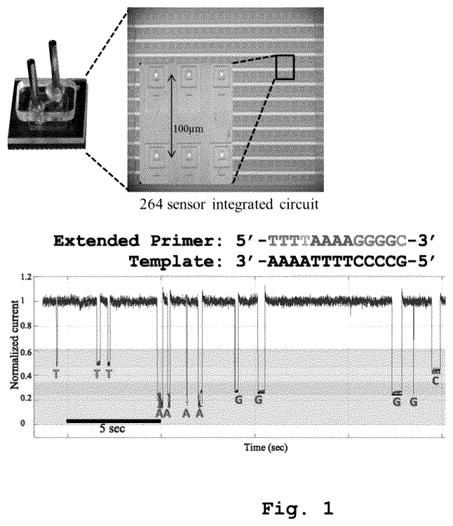

We have developed the SM electronic approach with nanopore arrays for DNA sequencing by synthesis (SBS)¹⁻³ and genotyping^{4,5} using oligonucleotide-based polymer tags. The tags were synthesized with a 5′-alkyne moiety which readily reacts with the desired azido-dN6P by azide-alkyne Huisgen cycloaddition⁷ to produce the tagged nucleotides. The tags consist of oligodeoxynucleotides (e.g., dT₃₀), in some cases interspersed with modified phosphodiester building blocks having specific base or backbone modifications, different tags for A, C, G and T. We also developed a CMOS-based nanopore array chip for SM detection of multiple polymer tags (FIG. 1, left). The chip consists of 264 individually-addressable wells comprising nanopores for real-time recording of fluctuations in the electric current with high temporal resolution.^{2,3} We have optimized methods for conjugating DNA polymerases to protein nanopores, formed complexes with DNA templates and primers and added the four tagged nucleotides to accomplish real-time SM electronic SBS. The platform performance is evident from the representative sequencing traces included in FIG. 1, right and in our publications,²³ where 4 current levels corresponding to 4 different polymer tags in addition to the open channel current are clearly distinguishable in the histograms, yielding reliable SM DNA sequencing data.

Thus, nanopores can be used as SM electronic detector for a variety of polymer tags. The details of the invention for detection of viral particles, viral antigens and viral antibodies are described as follows:

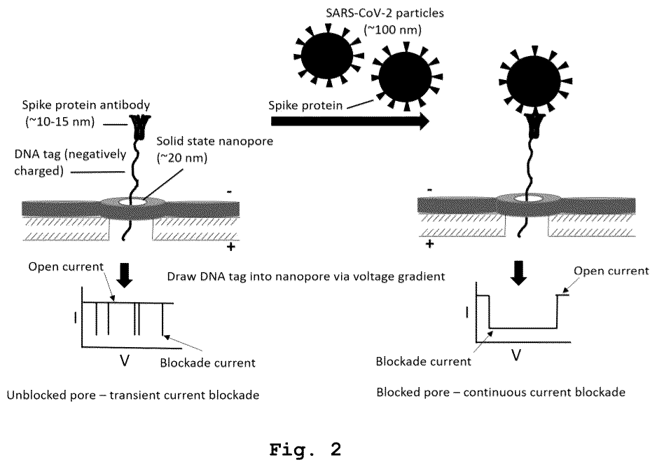

### EXAMPLE 1: ELECTRONIC SINGLE MOLECULE DETECTION OF INTACT SARS-COV-2 VIRAL PARTICLES IN ENVIRONMENTAL OR BIOLOGICAL SAMPLES (FIG. 2)

Using coronavirus as an example, the electronic SM detection of intact SARS-CoV-2 particles using DNA tagged antibodies to the trimeric spike (S) protein of intact viral particles is illustrated in FIG. 2. The platform comprises 2 components, a nanopore of ˜20 nm diameter as the SM electronic detector and a DNA-tagged antibody specific for the SARS-CoV-2 spike protein.

Intact aerosolized coronavirus particles or uncontaminated aerosolized dust particles (as background control) in the case of environmental samples are collected and concentrated using commercial air sampling technology into a buffer solution containing DNA-tagged antibodies that are specific to the S protein of SARS-CoV-2; the DNA tags, similar to those we used for nanopore-based SM electronic SBS, are polymeric molecules with small diameters that can easily enter nanopores. As shown in FIG. 2, right, the virus particles are captured by the DNA tagged antibodies. This solution is applied to chips containing solid-state nanopores, and a voltage is applied to attract the negatively charged DNA tags into the nanopore. In the case of tagged antibodies bound to the virus, the large viral particles (˜100 nm diameter) will plug the nanopores (˜20 nm diameter). This will result in the DNA tag being retained in the nanopore with the generation of a continuous ionic current blockade signal for as long as the voltage gradient is maintained (e.g., up to a second). Multiple such voltage pulses over several seconds to a minute will assure capture of sufficient virus attached antibodies and recording of sufficient events to achieve the desired sensitivity and specificity relative to a control solution with dust particles but no virus. In contrast, in the absence of bound viral particles, the DNA-tagged antibodies (10-15 nm diameter) will rapidly pass through the nanopore, resulting in only transient current blockade events (FIG. 2, left).

Concentration of the virus from the air is achieved using established technology involving centrifugal force or electrokinetic propulsion and elution into the buffer solution.

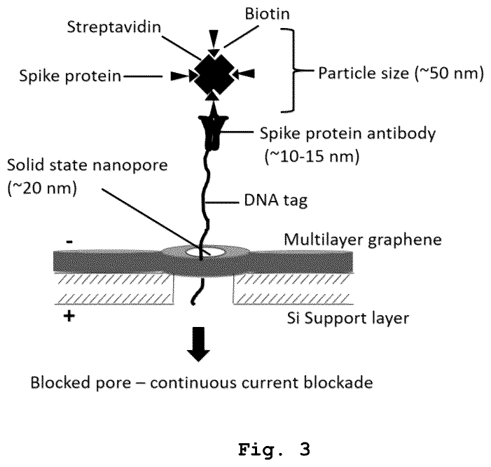

When adapting the system for new viral particles, in place of actual viral particles that require a BSL-3 facility, viral mimics are used to establish the protocol: in the current example to generate SARS-CoV-2 viral mimics, (1) Streptavidin is incubated with biotinylated Spike proteins (4 proteins attached to each Streptavidin molecule, FIG. 3), to generate particles which are also too large (˜50 nm) to enter the nanopore, and (2) pseudotype viruses (non-viable virus particles decorated with SARS-CoV-2 spike surface protein) to simulate more closely the size and other structural characteristics of infectious SARS-CoV-2. When viral mimics are not present, simulating background measurements, the DNA-tagged antibodies will rapidly pass through the nanopore, resulting in only transient current blockade events.

Because of the single molecule electronic digital nature of the detection, the technology is highly sensitive and quantitative. With just a few seconds of rapid recording, it is possible to achieve high specificity and sensitivity. In addition, the nanopore platform is envisioned as a low cost hand-held device that can be deployed in essentially any setting (businesses, hospitals, etc.), in combination with collectors to monitor for aerosol contamination, swabbing for surface fomites, or home test kits for sputum, nasal or blood samples. Moreover, the platform can be rapidly adapted for newly emerging viral pathogens, by simply switching the virus-specific antibody attached to the DNA tag.

The same detection protocol is used for biological samples, following the collection of nasal swabs, sputum or blood serum into buffers.

Though the order of addition of reagents indicated above (combining virus and tagged antibody before applying to the nanopore chip) is one embodiment, the virus-containing sample may be directly added to nanopore chips with DNA tagged antibodies already in the cis compartment above the nanopores prior to application of the voltage gradient.

In the above description, the target is SARS-CoV-2 and the capture/detection agent is DNA-tagged antibody specific for the Spike protein. However, the same procedure and platform can be used to identify other surface proteins (the envelope protein E and the membrane protein M) in SARS-CoV-2, as well as surface proteins specific for a wide variety of viruses. These comprise other coronaviruses such as SARS-CoV and MERS, various influenza virus types and strains, the human immunodeficiency virus HIV-1, hepatitis viruses A, B and C, Ebola virus, and other viruses that commonly infect humans, other animals (e.g., pets and livestock) and plants (e.g., food crops). The only difference in the procedure is the choice of antibody, which should be specific for a surface protein of the targeted virus. For optimization purposes, mimics of any of the above viruses can be prepared using either the biotin-streptavidin strategy or pseudoviruses with surface proteins of the virus of interest.

### EXAMPLE 1.1: CONJUGATION OF DNA TAG TO ANTIBODY

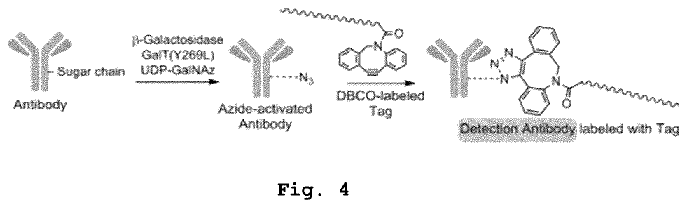

There are many established methods for conjugation of antibodies to proteins, drugs, oligonucleotides, or other biological targets,⁸ and many of these are commercially available as labeling kits. For instance, the SiteClick kit (Thermo Fisher) uses copper-free click chemistry between azide and dibenzocyclooctane (DBCO) (FIG. 4). In the first step of SiteClick conjugation, terminal galactose residues on the N-linked sugars in the Fc region on the antibody are removed by R-galactosidase. The azide containing sugar, UDP-GalNAz, is then added to the modified carbohydrate domain of the antibody via the Gal-1-P uridylyltransferase (Gal-T)-catalyzed reaction targeting the terminal GlcNAc residues. This specific targeting maintains the integrity of the antigen-binding site on the antibody. The DBCO-modified polymer tags (single-stranded DNA oligonucleotide) are made by reacting the 5′-amino-modified oligonucleotides with DBCO-NHS ester. The DBCO-modified oligonucleotide tags are then reacted with azido-modified antibody using copper-free click chemistry to provide antibody coupled with oligonucleotide tag. The oligonucleotide coupled antibody is purified by gel electrophoresis or FPLC.

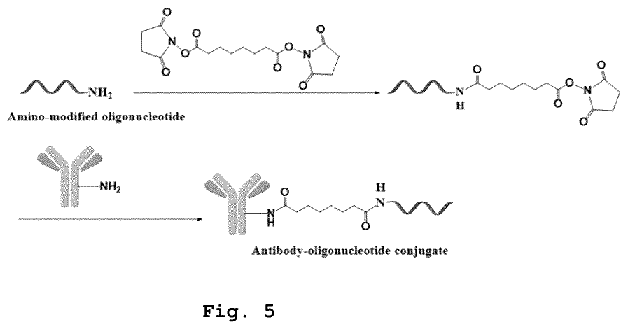

In another approach, the amino-containing oligonucleotide tag (ssDNA) is reacted with an excess of the homobifunctional linker disuccinimidyl suberate (DSS), and purified by high performance liquid chromatography (HPLC). The resulting activated-oligonucleotides are coupled to the antibodies (FIG. 5).

Antibodies are selected based on the literature. While antibodies directed against the prefusion state of the receptor binding domain of the SARS-CoV-2 spike protein are a preferred embodiment, there are numerous commercially available antibodies directed against other domains of the spike protein or other surface proteins (M and E) that can be used. Similar antibodies exist for surface proteins of other viruses.

### EXAMPLE 1.2: GENERATION OF STREPTAVIDIN-BASED PROTEIN PARTICLES AS VIRUS MIMICS

Excess biotinylated recombinant trimeric S protein (R&D Systems, Minneapolis, MN) or equivalent surface proteins from SARS-CoV-2 or other viruses are bound to Streptavidin, and complexes with 4 copies of the S protein are purified by FPLC and gel electrophoresis.

### EXAMPLE 1.3: TESTS WITH PSEUDOVIRUSES

A SARS-CoV-2 pseudotype virus has been constructed. This replication-deficient retrovirus expresses SARS-CoV-2 spike proteins on its surface.⁹ The virus can be used under BSL-2 conditions, circumventing experimentation at BSL-3 level as necessary for SARS-CoV-2. The advantage of the use of the pseudovirus over the previously described Streptavidin/S protein mimic is that it more closely resembles the structure of authentic SARS-CoV-2 viral particles. Other pseudoviruses can be generated with a variety of viral surface proteins of interest.

### EXAMPLE 1.4: CAPTURE/CONCENTRATION OF SARS-COV-2 SPIKE PROTEIN EXPRESSING PSEUDOVIRUS AND VIABLE SARS-COV-2

Different approaches for the collection of aerosolized viral particles exist. For instance, a BSL-3 set-up can be used to practice this invention, including alternative wet and dry cyclonic air samplers and a novel electrokinetic capture approach.¹⁰ Initial optimization tests can be run with the aerosolized pseudovirus expressing SARS-CoV-2 spike protein at various concentrations, then similar tests can be performed with SARS-CoV-2 aerosols.

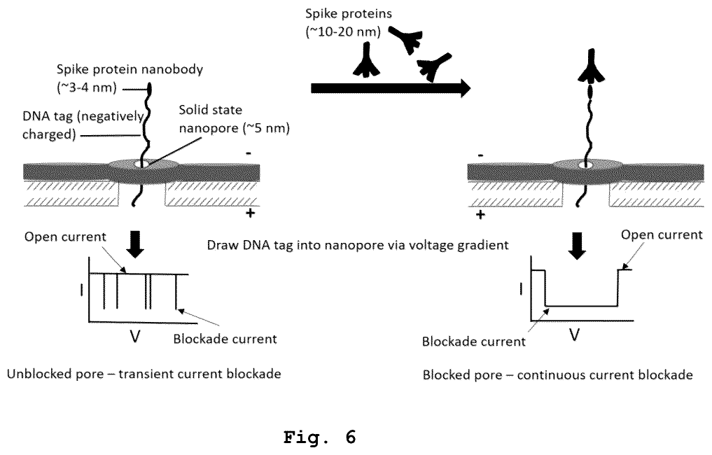

### EXAMPLE 2: ELECTRONIC SINGLE MOLECULE DETECTION OF SARS-COV-2 ANTIGENS IN ENVIRONMENTAL OR BIOLOGICAL SAMPLES (FIG. 6)

Using coronavirus as an example, a method to detect SARS-CoV-2 antigens, principally from biological samples, but potentially in environmental samples such as surfaces and wastewater streams, utilizing the single molecule electronic platform is described. Unlike in the preceding example, intact viruses are not required, and would not be the typical targets. The approach, presented in FIG. 6, utilizes DNA tagged nanobodies, the variable heavy chain domain of camelid-derived antibodies, which, in addition to their small size, display high affinity to their target antigens, high stability and solubility,^{11,12} all features that are advantageous for our single molecule electronic approach. In addition to nanobodies, one can also use other natural or synthetic DNA tagged antibody mimetics (e.g., affibodies,¹³ adnectins (monobodies),¹⁴ anticalins,¹⁵ affimers,¹⁶ and peptide or nucleic acid aptamers^{17,18}), all of which are substantially smaller than a full antibody (˜4 nm or less) and can be designed to have the desired specificity and relatively high affinity for their target antigen. The nanobodies and the other antibody mimetics can comprise a modified amino acid bearing a specific chemical group that allows facile conjugation to the DNA tag.

The basic protocol is indicated in FIG. 6. In brief, nasal, oropharyngeal or blood samples are obtained and added to a buffer solution containing DNA-tagged nanobodies that are specific to the S protein of SARS-CoV-2; the tags are polymeric molecules with a small diameter that can easily enter nanopores. As shown in FIG. 6, right, the Spike proteins attach to the DNA tagged nanobodies. This solution is applied to chips containing solid-state or protein nanopores, and a voltage is applied to attract the negatively charged DNA tags into the nanopore. In the case of virus-bound nanobodies, the S proteins (˜10-20 nm diameter) will plug the nanopores (in this case ˜5 nm diameter). This will result in the DNA tag being retained in the nanopore with the generation of a continuous ionic current blockade signal for as long as the voltage gradient is maintained (e.g., up to a second). Multiple such voltage pulses over several seconds to a minute will assure capture of sufficient S protein attached antibodies and recording of sufficient events to achieve the desired sensitivity and specificity relative to a control uninfected sample. In contrast, in the absence of bound S protein antigens, the DNA-tagged nanobodies (˜3-4 nm diameter) will rapidly pass through the nanopore, resulting in only transient current blockade events (FIG. 6, left).

Though the order of addition of reagents indicated above (combining antigens in sample and DNA tagged nanobody before introducing to the nanopore chip) is one embodiment, the viral antigen samples may be directly added to nanopore chips with tagged nanobodies already in the cis compartment above the nanopores prior to application of the voltage gradient.

In the above description, the target is the SARS-CoV-2 Spike protein and the capture/detection agent is DNA-tagged nanobody specific for the Spike protein. However, the same procedure and platform can be used to identify other structural proteins (the envelope protein E, the membrane protein M, the nucleocapsid protein N) in SARS-CoV-2, as well as structural proteins specific for a wide variety of alternative viruses. These comprise other coronaviruses such as SARS-CoV and MERS, various influenza virus types and strains, the human immunodeficiency virus HIV-1, hepatitis viruses A, B and C, Ebola virus, including viruses that commonly infect humans, other animals (e.g., pets and livestock) and plants (e.g., food crops). The only difference in the procedure is the choice of nanobody or other antibody mimetic, which should be specific for a structural protein of the targeted virus. Though not the primary purpose in this example, the approach can also be used to detect intact virus particles, so long as there is high affinity binding of the viral particle to the nanobody or other antibody mimetics, and this interaction can withstand the voltage gradient long enough to obtain convincing nanopore signals.

This approach can also be used to detect bacterial antigens or indeed any antigenic protein (for instance those released during disease processes including cancer biomarkers), as long as the capture molecule (DNA tagged nanobody or other antibody mimetic) is narrower than the diameter of the nanopore and the antigen target is larger than the nanopore diameter.

### EXAMPLE 2.1: ELECTRONIC SINGLE MOLECULE DETECTION OF MULTIPLE VIRAL ANTIGENS SIMULTANEOUSLY IN ENVIRONMENTAL OR BIOLOGICAL SAMPLES

It is often desirable to identify multiple viral antigens simultaneously. For instance, one might want to know whether a person is infected with a coronavirus or an influenza virus. In order to achieve this, the nanobodies specific for an influenza protein would be covalently linked to a different tag than the nanobodies specific for a coronavirus protein. Because of the narrow nanopore diameter in Example 2, modifications of the polymeric tag could elicit substantial differences in ionic current relative to open current readings. For example, four different polymer tags can be used to obtain 4 different nanopore current blockade signals;² if each is attached to a different antibody specific for a different viral antigen, 4 different antigens can be detected simultaneously. The synthesis and characterization of such tags is described below. Following incubation of the antigen-containing samples with the tagged nanobodies, the mixture is introduced to the nanopore chip. Following establishment of a voltage gradient, the different ion currents elicited in the nanopores determine which viral antigen was present in the sample. This multiplexing procedure can be used to detect different intact virus particles as well by using polymer tagged nanobodies or other antibody mimetics specific for surface proteins of the viral particles. The invention comprises single nanopore chips and nanopore array chips.

### EXAMPLE 2.2: ELECTRONIC SINGLE MOLECULE QUANTITATION OF VIRAL ANTIGENS SIMULTANEOUSLY IN ENVIRONMENTAL OR BIOLOGICAL SAMPLES

It is often desirable to track, for example, infectious disease antigens or cancer biomarkers over time or following treatment. In this case, one might want to quantify the amounts of these antigens at each stage of the infectious disease process, cancer progression or treatment. Here a variety of nanopore-distinguishable tags are attached to the nanobody or other antibody mimetic as described in Example 2.1 and each such uniquely tagged nanobody or antibody mimetic is incubated with a sample from a different time point. Using a nanopore array consisting of hundreds or thousands of nanopores, each of which can be interrogated independently, one can determine the percentage of nanopores exhibiting an ionic current signature specific to each tag. The tag present in the majority of places on the nanopore would indicate which sample had the highest relative antigen concentration. The approach can also be used to quantitate viral particles.

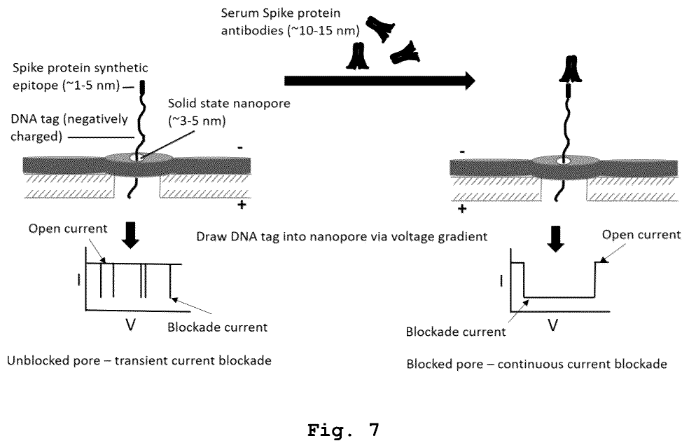

### EXAMPLE 3: ELECTRONIC SINGLE MOLECULE DETECTION OF SARS-COV-2 ANTIBODIES IN ENVIRONMENTAL OR BIOLOGICAL SAMPLES (FIG. 7)

During the course of an infectious disease, the host mounts cellular and humoral immune responses against components (proteins, carbohydrates and lipids) of the invading pathogen. The latter is typified by the production of antibodies of various isotypes (IgM, IgG, IgD, IgA, IgE) which are released in a temporal and tissue-specific manner. Typically, IgM antibodies appear early in an infectious disease process, with IgG and other forms appearing later. Antibodies are typically raised against small segments of the viral proteins called epitopes. Our single molecule electronic approach to detect SARS-CoV-2 antibodies as an example takes advantage of these features of the immune response. For the purpose of this example, we use synthetic epitopes of up to ˜20 amino acids in length which have the desired diameter (the ability to enter and traverse the nanopore). The synthetic epitopes can have a modified amino acid bearing a specific chemical group that allows facile conjugation to the DNA tag in the same way as described in Example 2 for attachment of DNA tags to nanobodies or other antibody mimetics.

The basic protocol is indicated in FIG. 7. In brief, serum samples are obtained and added to a buffer solution containing DNA-tagged synthetic epitopes that are specific to the antibodies to the S protein produced during the immune response; the tags are polymeric molecules that can easily enter nanopores. As shown in FIG. 7, right, the antibodies are captured by the DNA tagged synthetic epitopes. This solution is applied to chips containing solid-state or protein nanopores, and a voltage is applied to attract the negatively charged DNA tags into the nanopore. In the case of antibody-bound synthetic epitopes, the S protein antibodies (˜10-15 nm diameter for IgG) will plug the nanopores (in this case ˜3-5 nm diameter). This results in the DNA tag being retained in the nanopore with the generation of a continuous ionic current blockade signal for as long as the voltage gradient is maintained (e.g., up to a second). Multiple such voltage pulses over several seconds to a minute will assure capture of sufficient S protein antibody-attached epitopes and recording of sufficient events to achieve the desired sensitivity and specificity relative to a control uninfected sample. In contrast, in the absence of bound antibodies, the DNA-tagged synthetic epitopes (˜1-5 nm diameter) will rapidly pass through the nanopore, resulting in only transient current blockade events (FIG. 7, left).

Though the order of addition of reagents indicated above (combining antibodies in sample and DNA tagged synthetic epitopes before introducing to the nanopore chip) is one embodiment, the antibody containing sample may be directly added to nanopore chips with tagged antigenic epitopes already in the cis compartment above the nanopores prior to application of the voltage gradient.

Though in Example 3, synthetic epitopes are used as an exemplary case, they can be replaced with other small diameter specific antibody binding molecules including anti-idiotypic nanobodies, anti-idiotypic affibodies, anti-idiotypic affimers, etc. For distinguishing different isotypes (IgM, IgG, etc.), anti-isotypic nanobodies, anti-isotypic affibodies, anti-isotypic affimers, etc., can be used.

### EXAMPLE 3.1: ELECTRONIC SINGLE MOLECULE DETECTION OF MULTIPLE VIRAL ANTIBODIES SIMULTANEOUSLY IN ENVIRONMENTAL OR BIOLOGICAL SAMPLES

It is often desirable to identify multiple viral antibodies simultaneously. For instance, one might want to know whether a person has been previously infected with a coronavirus or an influenza virus. In order to achieve this, the synthetic epitopes or other antibody binding molecules specific for an influenza antibody would be covalently linked to a different tag than the synthetic epitopes or other antibody binding molecules specific for a coronavirus antibody. Because of the narrow nanopore diameter in Example 3, modifications of the polymeric tag could elicit substantial differences in ionic current relative to open current readings. For example, four different polymer tags can be used to obtain 4 different nanopore current blockade signals;² if each is attached to a different synthetic epitope specific for a different viral antibody, 4 different antibodies can be detected simultaneously. The synthesis and characterization of such tags is described below. Following incubation of the antibody-containing samples with the tagged synthetic epitopes or other antibody binding molecules, the mixture is introduced to the nanopore chip. Following establishment of a voltage gradient, the different ion currents elicited in the nanopores determine which viral antibody was present in the sample. The invention comprises single nanopore chips and nanopore array chips.

### EXAMPLE 3.2: ELECTRONIC SINGLE MOLECULE QUANTITATION OF VIRAL ANTIBODIES SIMULTANEOUSLY IN ENVIRONMENTAL OR BIOLOGICAL SAMPLES

It is often desirable to track, for example, infectious disease antibodies over time or following treatment. In this case, one might want to quantify the amounts of these antibodies at each stage of the infectious disease process, cancer progression or treatment. Here a variety of nanopore-distinguishable tags are attached to the synthetic epitope or other antibody binding molecules as described in Example 3.1 and each such uniquely tagged synthetic epitope or antibody binding molecule is incubated with a sample from a different time point. Using a nanopore array consisting of hundreds or thousands of nanopores, each of which can be interrogated independently, one can determine the percentage of nanopores exhibiting an ionic current signature specific to each tag. The tag present in the majority of places on the nanopore would indicate which sample had the highest relative antibody concentration.

### EXAMPLE 4: CONSTRUCTION OF NANOPORE CHIPS

Well established protocols exist to fabricate solid-state nanopores.¹⁹⁻²¹ For example, a graphene nanopore chip could be used for the viral particle/antigen/antibody detection approach described herein. Nanopores from 2-20 nm can be easily produced and used for the detection schemes in Examples 1-3. Chips with an array of individually addressable graphene nanopores can be manufactured similarly to the CMOS nanopore chip design in our prior publications.^{2,3} Such an array design would increase sensitivity of viral particle, antigen or antibody detection and allow for quantitation and even multiplexing if desired.

### EXAMPLE 5: CONJUGATION CHEMISTRIES TO ATTACH POLYMER TAGS TO CAPTURE AGENTS (ANTIBODIES, NANOBODIES AND OTHER ANTIBODY MIMETICS, SYNTHETIC ANTIGENIC EPITOPES)

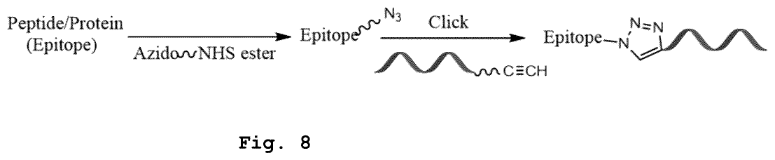

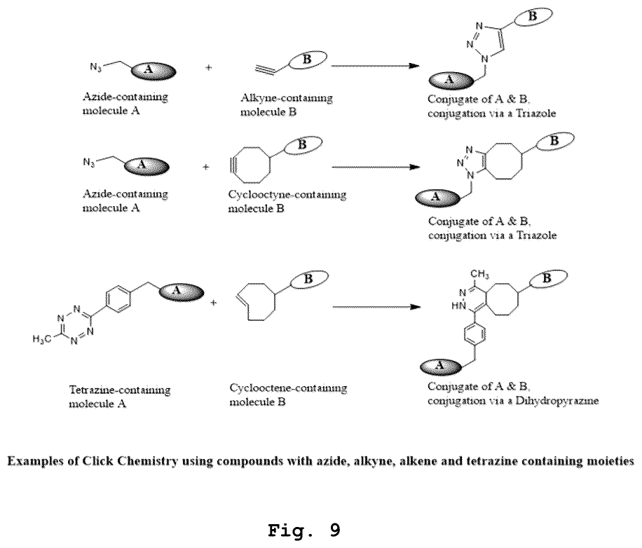

There are many established conjugation chemistries to attach antibodies, antibody mimetics or synthetic epitopes to other biological molecules such as proteins, drugs or oligonucleotides (e.g., DNA).²² An epitope is the specific part of the antigen to which antibodies bind. While the antigen evokes the antibody response in the host, the antibody doesn't bind to the entire protein, but only to a segment called an epitope. The epitope, typically less than 20 amino acids long, can be easily synthesized using a modified amino acid comprising an azido or alkynyl amino acid at the terminal end or internally; alternatively, the synthetic peptide can be easily modified with similar chemical moieties by coupling of an amino group (e.g., on a lysine) with azido or alkynyl-NHS esters. The modified synthetic epitope can be coupled with appropriately functionalized (azido or alkynyl) oligonucleotide tags (single or double stranded DNA) using Click chemistry as shown in FIG. 8. Other Click conjugation chemistries are shown in FIG. 9.

### EXAMPLE 6: SYNTHESIS OF POLYMER TAGS FOR COUPLING TO ANTIBODIES, NANOBODIES, ANTIBODY MIMETICS AND SYNTHETIC EPITOPES FOR NANOPORE DETECTION

Nanopore detectable tags comprise organic polymeric molecules which can be detected in nanopore under the applied electric current.^{2,23} Nanopore detectable tags comprise polyethylene glycol molecules (PEGs), peptides, carbohydrates, oligonucleotides, single or double stranded DNA, aromatic compounds of different bulk and diameter or a combination thereof.

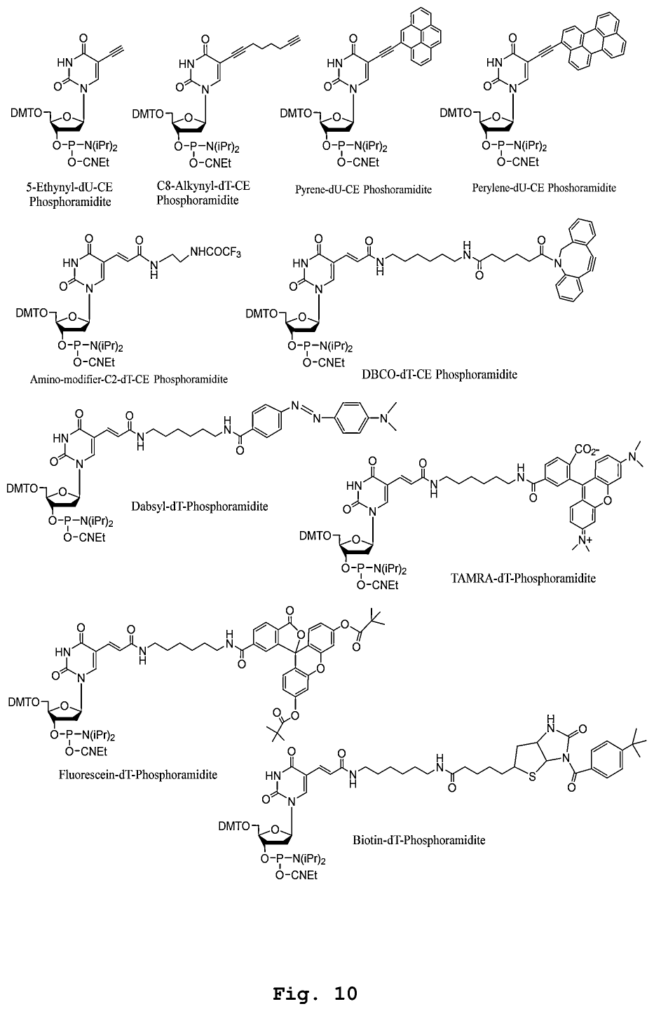

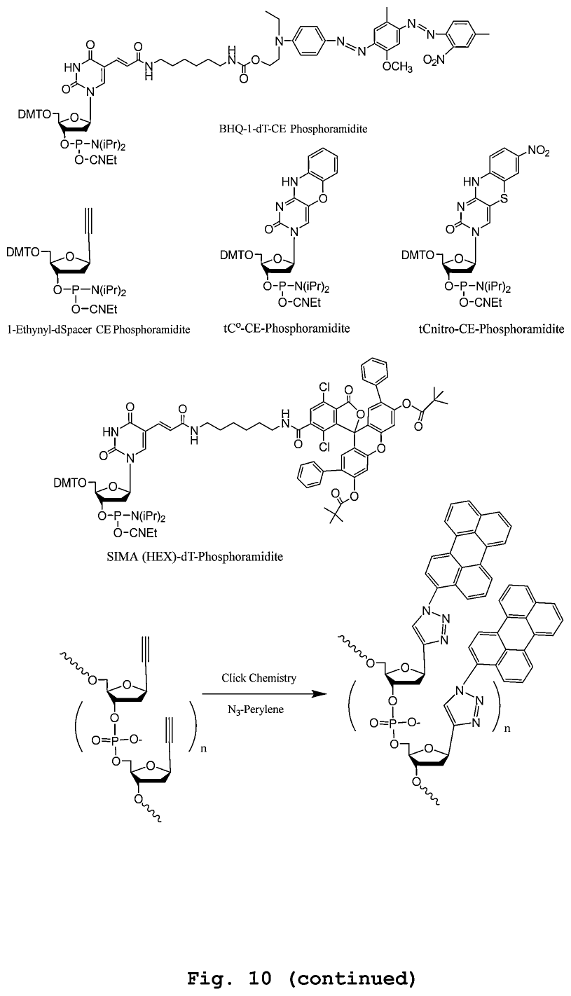

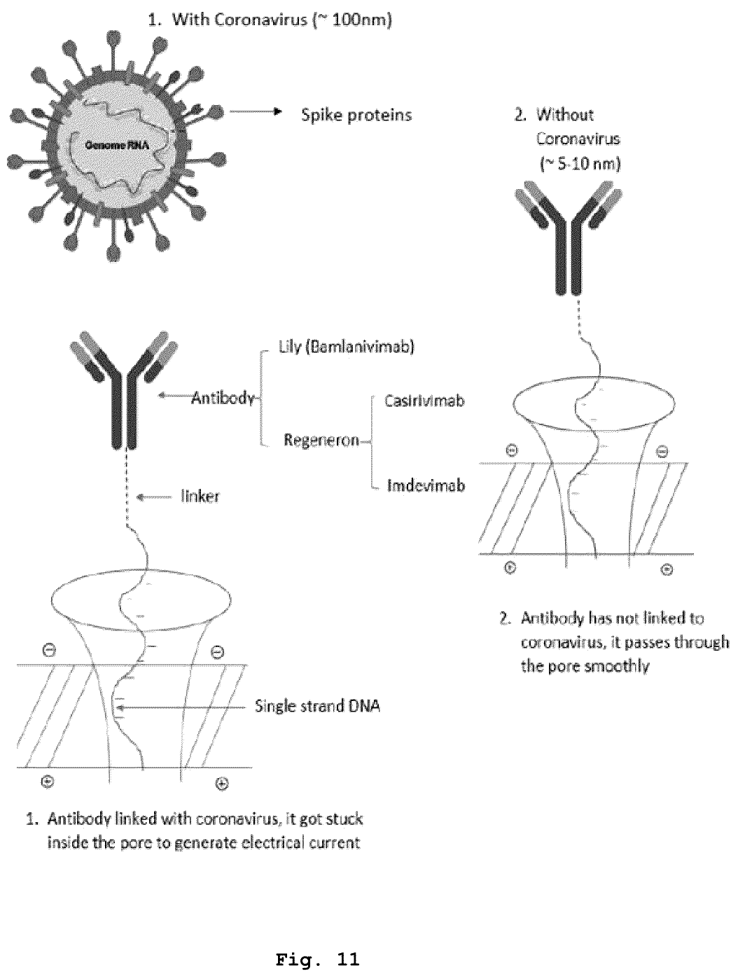

In a preferred embodiment, the polymeric nanopore detectable tags comprise oligonucleotides, either single stranded, double stranded, or with hairpin, triplex or quartet structures, of different length, charge or bulk. Oligonucleotides can be conveniently synthesized on a DNA synthesizer using standard phosphoramidites consisting of natural bases (A, T, G, C) or modified bases. The backbone of the oligonucleotides comprises phosphodiester, phosphorothioate, boranophosphate, methylphosphonate or other modifications. For multiplexing applications, oligonucleotides of different diameters can be synthesized using modified phosphoramidites available from Glen Research or other commercial suppliers. Some of these T/U modified phosphoramidites are shown in FIG. 10. These modified phosphoramidites comprise terminal amino groups, azido groups or alkynyl groups (for post synthetic modification) or bulky dyes or other aromatic bulky molecules. Oligonucleotides of different diameter with different bulky groups can be synthesized. The amino modified oligonucleotides can be conjugated post-synthetically to bulky dye-NHS esters or other bulky molecule-NHS esters of different size to provide different diameter oligonucleotides. Similarly, the oligonucleotides synthesized using alkynyl groups can be conjugated post-synthetically to bulky molecules containing azido groups. An example of such a modification is shown at the bottom of FIG. 10.

In some embodiments, the polymer tag is attached to the antibody/nanobody/antibody mimetic/synthetic antigen epitope using azide-alkyne Huisgen cycloaddtion, also known as “Click chemistry”. The azide-alkyne Huisgen cycloaddition is a 1,3-dipolar cycloaddition between an azide and a terminal or internal alkyne to give a 1,2,3-triazole as shown in FIG. 8.

In another embodiment, the oligonucleotide comprises 2-100 nucleotide base (either natural or modified) units. The length of the oligonucleotide should be long enough to reach the nanopore and generate appropriate current blockade under applied voltage. The oligonucleotides preferably comprise 5-50 bases and most preferably 10-30 bases.

### REFERENCES

1. Kumar S, Tao C, Chien M, Hellner B, Balijepalli A, Robertson J W F, Li Z, Russo J J, Reiner J E, Kasianowicz J J, Ju J. PEG-labeled nucleotides and nanopore detection for single molecule DNA sequencing by synthesis. Scientific Reports 2:684 (2012).
2. Fuller C W, Kumar S, Porel M, Chien M, Bibillo A, Stranges P B, Dorwart M, Tao C, Li Z, Guo W, Shi S, Korenblum D, Trans A, Aguirre A, Liu E, Harada E T, Pollard J, Bhat A, Cech C, Yang A, Arnold C, Palla M, Hovis J, Chen R, Morozova I, Kalachikov S, Russo J J, Kasianowicz J J, Davis R, Roever S, Church G M, Ju J. Real-time single-molecule electronic DNA sequencing by synthesis using polymer-tagged nucleotides on a nanopore array. Proceedings of the National Academy of Sciences USA 113:5233-5238 (2016).
3. Stranges P B, Palla M, Kalachikov S, Nivala J, Dorwart M, Trans A, Kumar S, Porel M, Chien M, Tao C, Morozova I, Li Z, Shi S, Aberra A, Arnold C, Yang A, Aguirre A, Harada E T, Korenblum D, Pollard J, Bhat A, Gremyachinskiy D, Bibillo A, Chen R, Davis R, Russo J J, Fuller C W, Roever S, Ju J, Church G M. Design and characterization of a nanopore-coupled polymerase for single-molecule DNA sequencing by synthesis on an electrode array. Proceedings of the National Academy of Sciences USA 113:E6749-E6756 (2016).
4. Tao C, Kumar S, Chien M, Ju J. Single Molecule Electronic Multiplex SNP Assay and PCR Analysis. U.S. patent Ser. No. 10/718,011.
5. Ju J, Cho Y, Kumar S, Kalachikov S, Tao C, Chien M, Russo J. Polymer tagged nucleotides for single molecule electronic SNP assay. U.S. patent Ser. No. 10/662,463.
6. Ju J, Kumar S, Tao C, Kalachikov, S, Russo J. Method for detecting multiple predetermined compounds in a sample. U.S. patent Ser. No. 10/732,183.
7. Rostovtsev V V, Green L G, Fokin V V, Sharpless K B. A stepwise Huisgen cycloaddition process: copper(I)-catalyzed regioselective “ligation” of azides and terminal alkynes. Angewandte Chemie International Edition English 41:2596-9 (2002).
8. Stephanopoulos N, Francis M B. Choosing an effective protein bioconjugation strategy. Nature Chemical Biology 7:876-884 (2011).
9. Schmidt F, Weisblum Y, Muecksch F, Hoffmann H H, Michailidis E, Lorenzi J C C, Mendoza P, Rutkowska M, Bednarski E, Gaebler C, Agudelo M, Cho A, Wang Z, Gazumyan A, Cipolla M, Caskey M, Robbiani D F, Nussenzweig M C, Rice C M, Hatziioannou T, Bieniasz P D. Measuring SARS-CoV-2 neutralizing antibody activity using pseudotyped and chimeric viruses. bioRxiv (2020).
10. Gordon J, Gandhi P, Shekhawat G, Frazier A, Hampton-Marcell J, Gilbert J A. A simple novel device for air sampling by electrokinetic capture. Microbiome 3:79 (2015).
11. Jovdevska I, Muyldermans S. The therapeutic potential of nanobodies. BioDrugs 34:11-26 (2020).
12. Bannas P, Hambach J, Koch-Nolte F. Nanobodies and nanobody-based human heavy chain antibodies as antitumor therapeutics. Frontiers in Immunology 8:1603 (2017).
13. Justino C I L, Duarte A C, Rocha-Santos T A P. Analytical applications of affibodies. Trends in Analytical Chemistry 65:73-82 (2015).
14. Lipovsek D. Adnectins: engineered target-binding protein therapeutics. Protein Engineering, Design and Selection 24:3-9 (2011).
15. Richter A, Eggenstein E, Skerra A. Anticalins: exploiting a non-Ig scaffold with hypervariable loops for the engineering of binding proteins. FEBS Letters 588:213-218 (2014).
16. Tiede C, Bedford R, Heseltine S J, Smith G, Wijetunga I, Ross R, AlQallaf D, Roberts A P E, Balls A, Curd A, Hughes R E, Martin H, Needham S R, Zanetti-Domingues L C, Sadigh Y, Peacock T P, Tang A A, Gibson N, Kyle H, Platt G W, Ingram N, Taylor T, Coletta L P, Manfield I, Knowles M, Bell S, Esteves F, Maqbool A, Prasad R K, Drinkhill M, Bon R S, Patel V, Goodchild S A, Martin-Fernandez M, Owens R J, Nettleship J E, Webb M E, Harrison M, Lippiat J D, Ponnambalam S, Peckham M, Smith A, Ferrigno P K, Johnson M, McPherson M J H, Tomlinson D C (2017) Affimer proteins are versatile and renewable affinity reagents. eLIFE 6:e24903.
17. Reverdatto S, Burz D S, Shekhtman A. Peptide aptamers: development and applications. Current Topics in Medicinal Chemistry 15:1082-1101 (2015).
18. Dunn M R, Jimenez R M, Chaput J C. Analysis of aptamer discovery and technology. Nature Reviews Chemistry 1:0076 (2017).
19. Niedzwiecki D J, Chou Y-C, Xia Z, Thei F, Drndid M. Detection of single analyte and environmental samples with silicon nitride nanopores: Antarctic dirt particulates and DNA in artificial seawater. Review of Scientific Instruments 91:031301 (2020).
20. Merchant C A, Healy K, Wanunu M, Ray V, Peterman N, Bartel J, Fischbein M D, Venta K, Luo Z, Johnson A T C, Drndid M. DNA translocation through graphene nanopores. Nano Letters 10:2915-2921 (2010).
21. Li R, Kothari M, Landauer A K, Cha M-H, Kwon H, Kim K-S. A new subcritical nanostructure of graphene-crinkle-ruga atructure and its novel properties. MRS Advances 3:2763-2769 (2018).
22. Hermanson G T, Bioconjugate Techniques 4^{th} edition. Academic Press.
23. Fuller C W, Kumar S, Ju J, Davis R, Chen R. Chemical methods for producing tagged nucleotides. U.S. patent Ser. No. 10/240,195.
24. Burnette W N. “‘Western blotting’: electrophoretic transfer of proteins from sodium dodecyl sulfate-polyacrylamide gels to unmodified nitrocellulose and radiographic detection with antibody and radioiodinated protein A”, Anal. Biochem. 1981; 112(2):195-203.
25. Engvall E, Perlmann P. “Enzyme-linked immunosorbent assay (ELISA), Quantitative assay of immunoglobulin”, G. Immunochemistry 1971; 8(9):871-874.
26. Yalow R S, Berson S A. “Immunoassay of endogenous plasma insulin in man”, J. Clin. Invest. 1960; 39:1157-1175.
27. Guerrera I C, Kleiner 0. “Application of mass spectrometry in proteomics”, Biosci. Rep. 2005; 25(1-2):71-93.
28. Gygi S P, Rist B, Gerber S A, Turecek F, Gelb M H, Aebersold R. “Quantitative analysis of complex protein mixtures using isotope-coded affinity tags”, Nat. Biotechnol. 1999; 17(10):994-999.
29. Klose J. “Protein mapping by combined isoelectric focusing and electrophoresis of mouse tissues: a novel approach to testing for induced point mutations in manimals”, Humangenetik 1975; 26(3):231-243.
30. O'Farrell P H. “High resolution two-dimensional electrophoresis of proteins”, J. Biol. Chem. 1975; 250(10):4007-4021.
40. Angenendt P., “Progress in protein and antibody microarray technology”, Drug Discov. Today 2005; 10(7):503-511.
41. Bussow K, Cahill D, Nietfeld W, Bancroft D, Scherzinger E, Lehrach H, Walter G. “A method for global protein expression and antibody screening on high-density filters of an arrayed cDNA library”, Nucleic Acids Res, 1998; 26(21):5007-5008.
42. Cahill D J. “Protein and antibody arrays and their medical applications”, J. Immunol. Methods, 2001; 250(1-2):81-91.
43. de Wildt R M, Mundy C R, Garick B D, Tomlinson I M. “Antibody arrays for high-throughput screening of antibody-antigen interactions”, Nat. Biotechnol, 2000; 18(9): 989-994.

1. A method of determining the presence of a viral particle in an environmental or biological sample comprising:
(a) contacting an environmental or biological sample potentially comprising the viral particle with an antibody having high affinity and specificity for a surface protein of said viral particle, wherein said antibody is covalently attached to a polymer tag, wherein the resulting polymer tagged antibody has substantially smaller dimensions than said viral particle,
(b) introducing resulting products obtained in (a) to a device comprising one or more nanopores, wherein the diameter of said nanopore(s) is larger than the dimensions of the polymer tagged antibody but smaller than the dimensions of the viral particle,
(c) applying a voltage gradient across the nanopore(s), in order to draw the polymer tag(s) into the nanopore(s),
(d) measuring current in the nanopore(s), wherein a continuous current blockade indicates binding of the viral particle to the polymer-tagged antibody, and wherein absence of a continuous blockade current will indicate absence of binding of the viral particle to the polymer-tagged antibody,
thereby determining the presence or absence of a viral particle in the sample; or a method of determining the presence of a viral antigen in an environmental or biological sample comprising:
(a) contacting an environmental or biological sample potentially comprising the viral antigen with a nanobody or an antibody mimetic with high affinity and specificity for said viral antigen, wherein said nanobody or antibody mimetic is covalently attached to a polymer tag, and wherein the resulting polymer tagged nanobody or polymer tagged antibody mimetic has substantially smaller dimensions than said viral antigen,
(b) introducing resulting products obtained in (a) to a device comprising one or more nanopores, wherein the diameter of said nanopore(s) is larger than the dimensions of the polymer tagged nanobody or polymer tagged antibody mimetic but smaller than the dimensions of the viral antigen,
(c) applying a voltage gradient across the nanopore(s), in order to draw the polymer tag(s) into the nanopore(s),
(d) measuring current in the nanopore(s), wherein a continuous current blockade indicates binding of the viral antigen to the polymer-tagged nanobody or polymer-tagged antibody mimetic, and wherein absence of a continuous blockade current will indicate absence of binding of the viral particle to the polymer-tagged antibody or polymer tagged antibody mimetic,
thereby determining the presence or absence of viral antigens in the sample.

2. The method of claim 1 wherein the viral particle comprises SARS-CoV-2, SARS-CoV, MERS, influenza A, influenza B, HIV-1, HBV, HCV and Ebola virus, and the polymer tagged antibody comprises an antibody with high affinity and specificity for a surface protein of SARS-CoV-2, SARS-CoV, MERS, influenza A, influenza B, HIV-1, HBV, HCV and Ebola virus.

3. (canceled)

4. The method of claim 1 wherein the viral antigen comprises an antigenic protein of SARS-CoV-2, SARS-CoV, MERS, influenza A, influenza B, HIV-1, HBV, HCV and Ebola virus, and the polymer tagged nanobody or polymer tagged antibody mimetic comprises a nanobody or antibody mimetic with high affinity and specificity for an antigenic protein of SARS-CoV-2, SARS-CoV, MERS, influenza A, influenza B, HIV-1, HBV, HCV and Ebola virus.

5. The method of claim 31, wherein instead of a viral antigen, the antigen comprises a bacterial antigen, a fungal antigen, a parasitic antigen, a tumor antigen, or another disease antigen, and the polymer tagged nanobody or polymer tagged antibody mimetic comprises a nanobody or antibody mimetic with high affinity and specificity for a bacterial antigen, a fungal antigen, a parasitic antigen, a tumor antigen, or another disease antigen.

6. The method of claim 1, wherein
a. the nanopore device comprises an array of nanopores,
b. more than one polymer tagged nanobody or polymer tagged antibody mimetic is used, each specific for a different antigen from the same or different viruses, wherein each said different tag for detecting each antigen comprises a different nanopore-distinguishable tag,
thereby determining which of said antigens is present in the sample.

7. The method of claim 1, wherein the antibody mimetic comprises an affibody, an adnectin, an anticalin, an affimer, a nucleic acid aptamer and a peptide aptamer.

8. A method of determining the presence of a virus-induced antibody in a biological sample comprising:
a. contacting a biological sample potentially comprising the viral-induced antibodie with a synthetic epitope with high affinity and specificity for said viral antibody, wherein said synthetic epitope is covalently attached to a polymer tag, and wherein the resulting polymer tagged synthetic epitope has substantially smaller dimensions than said viral antibody,
b. introducing resulting products obtained in (a) to a device comprising one or more nanopores, wherein the diameter of said nanopore(s) is larger than the dimensions of the polymer tagged synthetic epitope but smaller than the dimensions of the viral antibody,
c. applying a voltage gradient across the nanopore(s), in order to draw the polymer tag(s) into the nanopore(s),
d. measuring current in the nanopore(s), wherein a continuous current blockade indicates binding of the viral antigen to the polymer tagged synthetic epitope, and wherein absence of a continuous blockade current will indicate absence of binding of the viral antigen to the polymer tagged synthetic epitope,
thereby determining the presence or absence of virus induced antibodies in the sample.

9. The method of claim 8 wherein the viral antibody comprises an antibody elicited by infection with SARS-CoV-2, SARS-CoV, MERS, influenza A, influenza B, HIV-1, HBV, HCV and Ebola virus, and the polymer tagged synthetic epitope comprises a synthetic epitope with high affinity and specificity for an antibody elicited by SARS-CoV-2, SARS-CoV, MERS, influenza A, influenza B, HIV-1, HBV, HCV and Ebola virus infection.

10. The method of claim 8, wherein instead of a viral antibody, the antibody comprises an antibody elicited by a non-viral infection such as a bacterial infection, a fungal infection, or a parasitic infection, or comprises a tumor antibody or another disease antibody, and wherein the polymer tagged synthetic epitope comprises a synthetic epitope or antibody binding molecule with high affinity and specificity for an antibody elicited by a non-viral infection such as a bacterial infection, a fungal infection, or a parasitic infection, or a nanobody or antibody mimetics with a high affinity and specificity for a tumor antibody or another disease antibody.

11. The methods of claim 8 wherein, instead of the polymer tagged synthetic epitope, the polymer labeled synthetic antibody-binding molecule is a different type of molecule with high affinity and specificity for the antibody comprising anti-idiotypic nanobodies, anti-idiotoypic adnectins, anti-idiotypic anticalins, anti-idiotypic affibodies, anti-idiotypic affimers, anti-idiotypic nucleic acid aptamers and anti-idiotypic peptide aptamers.

12. The method of claim 8 wherein instead of the polymer tagged synthetic epitope, the polymer tagged synthetic epitope is a different type of molecule with high affinity and specificity for a particular class of antibodies comprising anti-isotypic nanobodies, anti-isotoypic adnectins, anti-isotypic anticalins, anti-isotypic affibodies, anti-isotypic affimers, anti-isotypic nucleic acid aptamer, and anti-isotypic peptide aptamers.

13. The method of claim 8, wherein
a. the nanopore device comprises an array of nanopores,
b. more than one polymer tagged synthetic epitope or other molecule with high affinity and specificity for the antibodies induced by the viral infection is used, each specific for a different antibody from the same or different viruses, wherein each said different synthetic epitope or other molecule with high affinity and specificity for the antibodies comprises a different nanopore-distinguishable tag,
thereby determining which of said antibodies is present in the sample.

14. The methods of claim 1, wherein the nanopore device comprises an array of nanopores.

15. A method for producing a SARS-CoV-2 viral mimic, comprising contacting Streptavidin with an excess of biotinylated trimeric SARS-CoV-2 Spike proteins, and isolation by gel electrophoresis or FPLC of high affinity complexes comprising 3 or 4 Spike proteins attached to Streptavidin, wherein said complexes have a larger diameter than Spike protein antibodies.

16. The method of claim 15 in which the biotinylated trimeric SARS-CoV-2 Spike protein is replaced by a different biotinylated surface protein of SARS-CoV-2 or a biotinylated surface protein of a different virus to produce a variety of viral mimics.

17. The method of claim 1 in which solutions containing the viral mimics produced in claims 15 and 16 are used in place of samples with intact viruses, thereby providing a test system for selection of antibodies and antigen targets.

18. The methods of claim 1, wherein the polymer-tagged antibody or other polymer-tagged molecules comprise oligonucleotide tags.

19. The method of claim 1, wherein the oligonucleotide tag comprises single stranded, double stranded, hairpin, triplex or quartet DNA molecules.

20. The method of claim 1, wherein the oligonucleotide tags have different diameters for multiplex determination of viral antigens or antibodies.
